(19) United States
(12) Patent Application Publication
(10) Pub. No.: US 2004/0137430 A1
(43) Pub. Date: Jul. 15, 2004

(54) Assays to detect or quantify bacterial or viral pathogens and contaminants

(75) Inventors: Dwight L. Anderson, Minneapolis, MN (US); Julio E. Sotillo Rodriguez, Plymouth, MN (US); Ron Anderson, Apple Valley, MN (US); Daniel W. Karl, St. Paul, MN (US); Michael C. Flickinger, Roseville, MN (US)

Correspondence Address: MUETING, RAASCH & GEBHARDT, P.A., P.O. BOX 581415, MINNEAPOLIS, MN, 55458, US

(73) Assignee: Regents of the University of Minnesota, Minneapolis, MN

(21) Appl. No.: 10/685,925

(22) Filed: Oct. 15, 2003

Related U.S. Application Data
(60) Provisional application No. 60/418,822, filed on Oct. 15, 2002.

(30) Foreign Application Priority Data
Oct. 15, 2003 (WO) PCT/US03/32641

(51) Int. Cl. C12Q 1/70; C12Q 1/04

(52) U.S. Cl. 435/5; 435/34

(57) ABSTRACT

The present invention provides methods for detecting or quantifying bacterial and viral pathogens or contaminants in a sample.

### CROSS-REFERENCE TO RELATED APPLICATIONS

The present invention claims priority to U.S. Provisional Application Serial No. 60/418,822, filed on Oct. 15, 2002, which is incorporated herein by reference.

### BACKGROUND

Bacterial and viral pathogens cause substantial morbidity and mortality among humans and domestic animals every year, as well as immense economic loss. Some, perhaps most, of this damage could be avoided if there were rapid and effective assays to detect and quantify the presence of these bacterial pathogens before they can cause widespread damage. For example, to mitigate the problems with food-borne pathogen infections, it would be beneficial to detect and quantify small numbers of pathogens that may be present in incoming ingredients, in-process materials, and final products used as a regular part of quality control and HACCP programs. Similarly, rapid detection and quantification of pathogens infecting an individual person and/or animal could not only improve the prognosis for the individuals, but could also be important in initiating steps to prevent or reduce the spread of the pathogen to other individuals. This invention is intended to meet this need.

The food-borne pathogens of greatest current concern to the food industry are Listeria monocytogenes, Salmonella spp., Campylobacter spp., and E. coli 0157/H7. These organisms are widespread contaminants that can cause fatal disease in susceptible individuals. Although all four are destroyed by thorough cooking, they pose a significant danger in uncooked foods, such as cheese, other dairy products, produce, juices, luncheon meats contaminated after cooking, and inadequately cooked meat.

Many pathogens are of commercial, medical, or veterinary concern. Such pathogens include, for example, gram-negative bacteria, including, for example, Campylobacter jejuni, Enterobacter spp., Klebsiella pneumoniae, and Salmonella typhi; gram-positive bacteria, including, for example, Bacillus spp., Clostridium perfringens, Staphylococcus aureus, and various Streptococcus spp.; mycoplasmas; and viruses. There is a need to rapidly detect and quantify such pathogens in a wide range of clinical samples, including, but not limited to, blood, sputum, cerebrospinal fluid, feces, and different types of swabs.

Traditional microbiological tests for these organisms rely on non-selective and selective enrichment cultures followed by plating on selective media and further testing to confirm suspect colonies. These procedures require several days. A variety of rapid methods have been investigated and introduced into practice to reduce the time requirement. Rapid techniques such as immunoassay or gene probes still typically require a biological enrichment step to achieve adequate sensitivity, a selective medium to achieve selectivity, or both since the intrinsic sensitivity of the best tests is the hundreds or thousands of cfu/ml. Polymerase chain reaction tests (PCR) include a biochemical amplification step and so are potentially capable of both very high sensitivity and selectivity. However, the sample size which can be economically subjected to PCR testing is limited. With dilute bacterial suspensions, most small subsamples will be free of cells, so PCR procedures still require enrichment steps. The time required for biological enrichment is dictated by the growth rate of the target bacterial population of the sample, by the effect of the sample matrix, and by the required sensitivity. For instance, a magnetic-capture PCR system for verotoxigenic E. coli requires 5, 7, and 10 hours enrichment to detect 1000, 100, and 1 cfu/ml, respectively, in a model system, and 15 hours enrichment to detect 1 cfu/g in ground beef. In practice, most high sensitivity methods employ an overnight incubation and take about 24 hours overall. Thus, there is a need for more efficient methods of detecting pathogenic bacteria and viruses.

### SUMMARY OF THE INVENTION

The present invention provides methods for detecting bacterial cells, bacteriophage, and viruses.

In one embodiment, the present invention provides a rapid and sensitive method of detecting a bacterial cell in a sample, the method including contacting the sample with bacteriophage including a binding agent, wherein the bacteriophage is specific to the bacterial cell; incubating the sample under conditions effective for the bacteriophage including a binding agent to infect the bacterial cell, and if a bacterial cell is present in the sample and has been infected by the bacteriophage including a binding agent, form new bacteriophage and release new bacteriophage into the sample, wherein the new bacteriophage do not include a binding agent; contacting the sample with a substrate including immobilized ligand for the binding agent under conditions effective for a complex to form between the bacteriophage including a binding agent and the substrate including immobilized ligand for the binding agent; removing the complexes of bacteriophage with a binding agent and substrate including immobilized ligand from the sample; and detecting new bacteriophage in the sample from which complexes have been removed, wherein the presence of new bacteriophage indicates the presence of a bacterial cell specific for the bacteriophage in the sample and wherein the absence of new bacteriophage indicates the absence of a bacterial cell specific for the bacteriophage in the sample. As used herein, “bacteriophage” includes one or more of a plurality of bacteriophages.

In another embodiment, the present invention provides a rapid and sensitive method of detecting a bacteriophage or virus in a sample, the method including contacting the sample with a substrate including immobilized binding agent specific for the bacteriophage or virus, under conditions effective for a complex to form between the bacteriophage or virus and the substrate including immobilized binding agent for the bacteriophage or virus; removing the complexes of bacteriophage or virus and substrate including immobilized binding agent for the bacteriophage or virus; and detecting the complexes of bacteriophage or virus and substrate including immobilized binding agent for the bacteriophage or virus, wherein the presence of complexes of bacteriophage or virus and substrate including immobilized binding agent for the bacteriophage or virus indicates the presence of a bacteriophage or virus in the sample and wherein the absence of complexes of bacteriophage or virus and substrate including immobilized binding agent for the bacteriophage or virus indicates the absence of bacteriophage or virus in the sample.

In another embodiment, the present invention provides a rapid and sensitive method of detecting a bacterial cell in a sample, the method including contacting the sample with bacteriophage including a binding agent, wherein the bacteriophage is specific to the bacterial cell; incubating the sample under conditions effective for the bacteriophage including a binding agent to infect the bacterial cell, and if a bacterial cell is present in the sample and has been infected by the bacteriophage including a binding agent, form new bacteriophage and release new bacteriophage into the sample, wherein the new bacteriophage do not include a binding agent; contacting the sample with a substrate including immobilized ligand for the binding agent under conditions effective for a complex to form between the bacteriophage including a binding agent and the substrate including immobilized ligand for the binding agent; removing the complexes of bacteriophage including a binding agent and substrate including immobilized ligand from the sample; contacting the sample with a second substrate including immobilized binding agent specific for the new bacteriophage, under conditions effective for a complex to form between the new bacteriophage and the second substrate including immobilized binding agent for the new bacteriophage; removing the complexes of new bacteriophage and second substrate including immobilized binding agent for the new bacteriophage; and detecting the complexes of new bacteriophage and second substrate including immobilized binding agent for the new bacteriophage, wherein the presence of complexes of new bacteriophage and second substrate including immobilized binding agent for the new bacteriophage indicates the presence of a bacterial cell specific for the bacteriophage in the sample and wherein the absence of complexes of new bacteriophage and second substrate including immobilized binding agent for the new bacteriophage indicates the absence of a bacterial cell specific for the bacteriophage in the sample.

In another embodiment, the present invention provides a rapid and sensitive method of detecting a bacterial cell in a sample, the method including combining a bacteriophage specific to the bacteria cell with the sample under conditions effective for the bacteriophage to infect the bacterial cell if present in the sample; contacting the sample with a first substrate including a first immobilized binding agent, under conditions effective for any bacteriophage that have not infected a bacterial cell to bind to the first immobilized binding agent; removing the first substrate and any bound bacteriophage; incubating the sample under conditions effective to form new bacteriophage within an infected bacterial cell and to release the new bacteriophage into the sample; contacting the sample with a second substrate including a second immobilized binding agent under conditions effective for the new bacteriophage, if present, to bind to the second immobilized binding agent; and detecting new bacteriophage bound to the second substrate including a second immobilized binding agent, wherein the presence of bound bacteriophage indicates the presence of a bacterial cell specific for the bacteriophage in the sample and wherein the absence of bound bacteriophage indicates the absence of a bacterial cell specific for the bacteriophage in the sample.

In another embodiment, the present invention provides a rapid and sensitive method of method of detecting a bacterial cell in a sample, the method including contacting the sample with a first substrate including immobilized bacteriophage specific to the bacterial cell; incubating the sample while in the presence of the first substrate under conditions effective for the immobilized bacteriophage to infect the bacterial cell, and if a bacterial cell is present in the sample and has been infected by the immobilized bacteriophage, form new bacteriophage and release new bacteriophage into the sample; contacting the sample with a second substrate including immobilized reporter cells under conditions effective for the new bacteriophage, if present, to infect the reporter cells; and detecting reporter cells infected by new bacteriophage, wherein the presence of reporter cells infected by new bacteriophage indicates the presence of a bacterial cell specific for the bacteriophage in the sample and wherein the absence of reporter cells infected by new bacteriophage indicates the absence of a bacterial cell specific for the bacteriophage in the sample. The first substrate and second substrate may be on the same substrate.

In another embodiment, the present invention provides a rapid and sensitive method of detecting a bacterial cell in a sample, the method including combining a bacteriophage specific to the bacteria cell with the sample under conditions effective for the bacteriophage to infect the bacterial cell if present in the sample; contacting the sample with a first substrate including a first immobilized binding agent under conditions effective for any bacteriophage that have not infected a bacterial cell to bind to the first immobilized binding agent; removing the first substrate and any bound bacteriophage; incubating the sample under conditions effective to form new bacteriophage within an infected bacterial cell and to release the new bacteriophage into the sample; contacting the sample with a second substrate including immobilized reporter cells under conditions effective for the new bacteriophage, if present, to infect the reporter cells; and detecting reporter cells infected by the new bacteriophage, wherein the presence of reporter cells infected by new bacteriophage indicates the presence of a bacterial cell specific for the bacteriophage in the sample and wherein the absence of reporter cells infected by new bacteriophage indicates the absence of a bacterial cell specific for the bacteriophage in the sample.

In another embodiment, the present invention provides a rapid and sensitive method of concentrating bacteriophage or virus in a sample, the method including contacting the sample with a substrate including immobilized binding agent specific for the bacteriophage or virus; incubating the sample under conditions effective for a complex to form between the bacteriophage or virus and the substrate including immobilized binding agent for the bacteriophage or virus; and allowing the complexes of bacteriophage or virus and the substrate including immobilized binding agent for the bacteriophage or virus to settle, thereby concentrating the bacteriophage or virus. The method may include further concentrating the sample by magnetic separation or centrifugation. The substrate including an immobilized binding agent specific for the bacteriophage or virus may be a bead with an iron core.

In another embodiment, the present invention provides a kit for detecting a bacterial cell in a sample, the kit including a porous substrate including immobilized bacteriophage specific to the bacterial cell and bacterial growth media. A porous substrate may include, for example, fibers, a fibrous filter, a membrane filter, and porous particles. The kit may also include printed instructions. The kit may also include one or more positive controls, one or more negative controls, one or more aliquots of magnetic polystyrene, streptavidin-coated beads, one or more aliquots of magnetic, polystyrene, antibody-coated beads, one or more aliquots of biotin-antibody-streptavidin-QUANTUM DOT complexes, one or more aliquots of magnetic polystyrene beads coated with protein G and complexed with specific antibody against the bacteriophage, one or more thin coverslip slides with detachable hollow cylinders mounted over magnetic needles, bibulous paper strips, and combinations thereof.

In another embodiment, the present invention provides a rapid and sensitive method of quantifying bacterial cells in a sample, the method including contacting the sample with bacteriophage including a binding agent, wherein the bacteriophage is specific to the bacterial cell; incubating the sample under conditions effective for the bacteriophage including a binding agent to infect the bacterial cell, and if a bacterial cell is present in the sample and has been infected by the bacteriophage including a binding agent, form new bacteriophage and release new bacteriophage into the sample, wherein the new bacteriophage do not include a binding agent; contacting the sample with a substrate including immobilized ligand for the binding agent under conditions effective for a complex to form between the bacteriophage including a binding agent and the substrate including immobilized ligand for the binding agent; removing the complexes of bacteriophage including a binding agent and substrate including immobilized ligand from the sample; and quantifying new bacteriophage in the sample from which complexes have been removed, wherein the number of new bacteriophage indicates the number of a bacterial cell specific for the bacteriophage in the sample.

In another embodiment, the present invention provides a rapid and sensitive method of quantifying bacteriophage or virus in a sample, the method including contacting the sample with a substrate including immobilized binding agent specific for the bacteriophage or virus, under conditions effective for a complex to form between the bacteriophage or virus and the substrate including immobilized binding agent for the bacteriophage or virus; removing the complexes of bacteriophage or virus and substrate including immobilized binding agent for the bacteriophage or virus; and quantifying the complexes of bacteriophage or virus and substrate including immobilized binding agent for the bacteriophage or virus, wherein the number of complexes of bacteriophage or virus and substrate including immobilized binding agent for the bacteriophage or virus indicates the number of bacteriophage or virus in the sample.

In another embodiment, the present invention provides a rapid and sensitive method of method of quantifying bacterial cells in a sample, the method including contacting the sample with bacteriophage including a binding agent, wherein the bacteriophage is specific to the bacterial cell; incubating the sample under conditions effective for the bacteriophage including a binding agent to infect the bacterial cell, and if a bacterial cell is present in the sample and has been infected by the bacteriophage including a binding agent, form new bacteriophage and release new bacteriophage into the sample, wherein the new bacteriophage do not include a binding agent; contacting the sample with a substrate including immobilized ligand for the binding agent under conditions effective for a complex to form between the bacteriophage including a binding agent and the substrate including immobilized ligand for the binding agent; removing the complexes of bacteriophage including a binding agent and substrate including immobilized ligand from the sample; contacting the sample with a second substrate including immobilized binding agent specific for the new bacteriophage, under conditions effective for a complex to form between the new bacteriophage and the second substrate including immobilized binding agent for the new bacteriophage; removing the complexes of new bacteriophage and second substrate including immobilized binding agent for the new bacteriophage; and quantifying the complexes of new bacteriophage and second substrate including immobilized binding agent for the new bacteriophage, wherein the number of complexes of new bacteriophage and second substrate including immobilized binding agent for the new bacteriophage indicates the number of bacterial cells specific for the bacteriophage in the sample.

In another embodiment, the present invention provides a rapid and sensitive method of quantifying bacterial cells in a sample, the method including combining a bacteriophage specific to the bacteria cell with the sample under conditions effective for the bacteriophage to infect the bacterial cell if present in the sample; contacting the sample with a first substrate including a first immobilized binding agent, under conditions effective for any bacteriophage that have not infected a bacterial cell to bind to the first immobilized binding agent; removing the first substrate and any bound bacteriophage; incubating the sample under conditions effective to form new bacteriophage within an infected bacterial cell and to release the new bacteriophage into the sample; contacting the sample with a second substrate including a second immobilized binding agent under conditions effective for the new bacteriophage, if present, to bind to the second immobilized binding agent; and quantifying new bacteriophage particles bound to the second substrate including a second immobilized binding agent, wherein the number of bound bacteriophage indicates the number of bacterial cells specific for the bacteriophage in the sample.

In another embodiment, the present invention provides a rapid and sensitive method of quantifying bacterial cells in a sample, the method including contacting the sample with a first substrate including an immobilized bacteriophage specific to the bacterial cell; incubating the sample while in the presence of the first substrate under conditions effective for the immobilized bacteriophage to infect the bacterial cell, and if a bacterial cell is present in the sample and has been infected by the immobilized bacteriophage, form new bacteriophage and release new bacteriophage into the sample; contacting the sample with a second substrate including immobilized reporter cells under conditions effective for the new bacteriophage, if present, to infect the reporter cells; and quantifying reporter cells infected by new bacteriophage, wherein the number of reporter cells infected by new bacteriophage indicates the number of a bacterial cells specific for the bacteriophage in the sample.

In another embodiment, the present invention provides a rapid and sensitive method of quantifying bacterial cells in a sample, the method including combining a bacteriophage specific to the bacteria cell with the sample under conditions effective for the bacteriophage to infect the bacterial cell if present in the sample; contacting the sample with a first substrate including a first immobilized binding agent under conditions effective for any bacteriophage that have not infected a bacterial cell to bind to the first immobilized binding agent; removing the first substrate and any bound bacteriophage; incubating the sample under conditions effective to form new bacteriophage particles within an infected bacterial cell and to release the new bacteriophage particles into the sample; contacting the sample with a second substrate including a second immobilized binding agent under conditions effective for the new bacteriophage particles, if present, to bind to the second immobilized binding agent; and quantifying the bacteriophage bound to the second substrate including a second immobilized binding agent, wherein the number of bound bacteriophage indicates the number of bacterial cells specific for the bacteriophage in the sample.

In another embodiment, the present invention provides a rapid and sensitive method quantifying bacterial cells in a sample, the method including combining a bacteriophage specific to the bacteria cell with the sample under conditions effective for the bacteriophage to infect the bacterial cell if present in the sample; contacting the sample with a first substrate including a first immobilized binding agent under conditions effective for any bacteriophage that have not infected a bacterial cell to bind to the first immobilized binding agent; removing the first substrate and any bound bacteriophage; incubating the sample under conditions effective to form new bacteriophage within an infected bacterial cell and to release the new bacteriophage into the sample; contacting the sample with a second substrate including immobilized reporter cells under conditions effective for the new bacteriophage, if present, to infect the reporter cells; and quantifying reporter cells infected by the new bacteriophage, wherein the number of reporter cells infected by new bacteriophage indicates the number of bacterial cells specific for the bacteriophage in the sample.

In the methods and kits of the present invention, the bacterial cell may be a food pathogen, including, but not limited to, Listeria monocytogenes, Salmonella spp., Campylobacter spp. and E. coli O157/H7. The bacterial cell may also be a pathogen of medical or veterinary significance or a bacterial cell of commercial significance.

In the methods and kits of the present invention, immobilized binding agents may be, but are not limited to, an antibody, biotin, streptavidin, a viral receptor protein, or a cell.

In the methods and kits of the present invention, the first substrate and the second substrate may be, but are not limited to, a bead (e.g., a polystyrene bead or a magnetic bead), a polymeric material (e.g., a latex coating), a filter (e.g., a membrane filter or a fiber filter), a free fiber, or a porous (e.g., apertured) membrane. Bacteriophage may be detected by staining with a fluorescent dye, including, but not limited to, ALEXA dye, or with fluorescent nanocrystals. Bacteriophage may be detected by visualization under a light microscope. In some embodiments, visualization by light microscope may take place after concentration of the bacteriophage, by using a flow system or scanning system to insure that the entire sample passes under the objective, by using a wide area imaging system, or by using a surface fluorometer. Reporter cells infected by bacteriophage may be detected by the incorporation of a bacterial luciferase coding sequence or a green fluorescence protein (GFP) coding sequence into the bacteriophage.

### DETAILED DESCRIPTION OF ILLUSTRATIVE EMBODIMENTS OF THE INVENTION

The following methods offer an important improvement to existing methods for the detection and quantification of bacterial cells and viruses, including food pathogens, such as Listeria, E. coli, Salmonella, and Campylobacter, and medical pathogens, such as Bordetella pertusiss, Chlamydia pneumoniae, and Mycoplasma pneumoniae.

The methods of the present invention provide high detection sensitivity in a short time without the need for traditional biological enrichment. For example, the present methods can provide for the detection or quantification of less than about 100, less than about 50 or less than about 10 bacterial cells or viruses in a sample. Preferably the present methods can provide for the detection or quantification of less than about 5, less than about 4, less than about 3, or less than about 2 bacterial cells or viruses in a sample. Most preferably, the methods of the present invention can provide for the detection and quantification of a single bacterial cell or virus in a sample.

The methods of the present invention allow for the rapid detection and quantification of bacterial cells or viruses. For example, the methods of the present invention can be performed in less than about ten hours to less than about twelve hours, more preferably in less than about four hours to less than about three hours, and most preferably in about two hours or less.

The methods of the present invention can accommodate a wide range of samples sizes. For example, samples as large as about 25 grams (gm) or about 25 milliliter (ml) may be used. Preferably, samples of about 1 gram (gm) or about 1 ml or less may be used. If necessary, prior to an assay, samples may be concentrated to reduce the sample volume.

Also, included in the methods of the present invention are methods based on phage amplification that overcome the need to kill the extra phage particles in the initial test solutions, such as is required in the methods of U.S. Pat. Nos. 5,723,330, 5,498,525, 5,447,836 and 4,797,363.

Bacterial Cells

Any bacterial cell for which a bacteriophage that is specific for the particular bacterial cell has been identified can be detected by the methods of the present invention. Those skilled in the art will appreciate that there is no limit to the application of the present methods other than the availability of the necessary specific phage/target bacteria. Bacterial cells detectable by the present invention include, but are not limited to, bacterial cells that are food pathogens. Bacterial cells detectable by the present invention include, but are not limited to, all species of Salmonella, all species of E. coli, including, but not limited to E. coli 0157/H7, all species of Listeria, including, but not limited to L. monocytogenes, and all species of Campylobacter. Bacterial cells detectable by the present invention include, but are not limited to, bacterial cells that are pathogens of medical or veterinary significance. Such pathogens include, but are not limited to, Bacillus spp., Bordetella pertusiss, Camplyobacter jejuni, Chlamydia pneumoniae, Clostridium perfringens, Enterobacter spp., Klebsiella pneumoniae, Mycoplasma pneumoniae, Salmonella typhi, Staphylococcus aureus, and Streptococcus spp. Cultures of all bacterial cells can be obtained, for example, from American Type Culture Collection (ATCC, P.O. Box 1549, Manassas, Va., USA). Bacterial cells detectable by the present invention also include, but are not limited to, contaminating bacterial cells found in systems of commercial significance, such as those used in commercial fermentation industries, ethanol production, antibiotic production, wine production, etc. Such pathogens include, but are not limited to, Lactobacillus spp. and Acetobacter spp. during ethanol production. Other examples of bacteria include those listed in W. Levinson et al., Medical Microbiology & Immunology, McGraw-Hill Cos., Inc., 6^{th} Ed., pages 414-433 (2000). All bacterial cultures are grown using procedures well known in the art.

Bacteriophage

Bacteriophage, also called phage, are highly selective for their hosts. Bacteriophage typing is useful at the species and strain level for identifying bacteria, for instance, in epidemiological investigation of food-borne illness. The specificity of a phage for its host is determined at two levels. Each phage has a host receptor that for tailed phage typically recognizes elements of the phage baseplate and phage tail fibers. Interaction of these components with complementary elements on the bacterial cell surface determines the ability of the phage to bind to the cell and inject its DNA. Enzymatic activity of baseplate elements is sometimes but not always required. There is substantial evidence that phage breeding, genetic engineering of fiber elements, and hybridization, can alter phage specificity at this level. The second level of control over specificity is the events occurring within the bacterial cell, after injection of the phage DNA. Factors that can impact the phage's effectiveness include the presence of restriction enzyme systems in the host and the presence or absence of corresponding protective modifications of the phage DNA, the presence of immunity repressors, and the ability of phage promoters and accessory proteins to co-opt the host RNA polymerase to make phage proteins. Immunity repressors result from the presence of closely related integrated prophages in the target genome and are typically of narrow specificity. Restriction systems and promoter specificity have similar effects on phage expression and plasmid expression, the latter being fairly well understood.

Besides exhibiting specificity, phages have the ability to produce a substantial amplification in a short time. Under optimum infection and host growth medium conditions, a given phage/bacterium combination gives rise to a consistent number of phage progeny. Generally, the lytic infection cycle produces 100 or more progeny phage particles from a single infected cell in about one hour. However, there are exceptions. For example, phi29 of B. subtilis is a premier phage system for study of morphogenesis because it gives a burst of 1,000 in a 35-minute life cycle. Bacteria can be multiply infected by phages (multiplicity of infection, m.o.i.), and the phage “burst” (progeny produced per cell) depends on the multiplicity. To produce high yields, a m.o.i. of 10 is generally used. Within an assay it may be necessary to include control comparison standards, done in the same medium, with known numbers of phages infecting known numbers of substrate-bound target cells.

For the detection of a given bacterial cell, a bacteriophage that is capable of infecting the bacterial cell, replicating within the bacterial cell and lysing the bacterial cell is selected. For any given bacterial cell a wide variety of bacteriophages are available, for example, from ATCC or by isolation from natural sources that harbor the host cells. The bacteriophage should also exhibit specificity for the bacterial cell. A bacteriophage is specific for a bacterial cell when it infects the given bacterial cell and does not infect bacterial cells of other species or strains. For the detection of a particular bacterial cell, one would also preferably select a bacteriophage that gives an optimal or maximal burst size.

The range of bacterial cells that can be detected by the present invention is limited only by the availability of a bacteriophage specific for the bacterial cell and will be realized to be vast by those skilled in the art. For example a list of phage types available from ATCC is published by them as the Catalogue of Bacteria & Bacteriophages and is available on the worldwide web at atcc.org. Other such depositories also publish equivalent data in their catalogues, and this may be used to identify possible bacteriophage reagents for the methods of the present invention.

Examples of specific bacteria/bacteriophage pairings include T4, which is specific for E. coli (Molecular Biology of Bacteriophage T4, 1994, J. D. Karam, ed., ASM Press), and Listeria monocytogenes phage A511, which is specific for L. monocytogenes (see, Loessner et al., Applied and Environmental Microbiology 62:1133, 1996). Over fourteen different Campylobacter phages are available from ATCC. A number of these are specific for C. jejuni and C. coli and form the basis for a bacteriophage typing system (J. Clin. Microbiol. 22:13-18, 1985). ATCC lists over twenty-four different phages specific for Salmonella; included is phi29, a well-studied phage for Salmonella typhimurium (Zinder, N. D. and Lederberg, J., J. Bacteriology 64:679-699, 1952).

High titer bacteriophage stocks are produced on an appropriate host cell strain by procedures well known in the art. For example, plate or broth lysis methods may be used in the production of high titer stocks of bacteriophage. The culture of many other bacteria/bacteriophage pairings is well known to those of skill in the art. See, for example, U.S. Pat. Nos. 5,679,510; 5,714,312; 5,858,648; 5,914,240; 5,985,596; 5,958,675; 6,090,541; 6,165,710; 6,190,856 B1; 6,203,996 B 1; 6,355,445 and 6,379,908. See also, for example, Bacteriophages, Mark Adams, InterSciences Publishers, Inc., New York, (1959) and “Phenotypic Characteristics of Coagulase-Negative Staphylococci: Typing and Antibiotic Susceptibility,” thesis of Jens Otto Jarlov, (1999), APMS Supplement, No. 91, Vol. 107.

Viruses

Viruses that can be removed using certain methods of the present invention include a wide variety of well-known viruses. These include those viruses that infect eukaryotic cells, particularly mammalian, and more particularly human cells. These include, but are not limited to, poliovirus, coxsackievirus, hepatitis A, B, and C viruses, smallpox virus, norwalk virus, rotavirus, rhinovirus, herpes simplex viruses, varicella-zoster virus, cytomegalovirus, and the like.

Methods of Detection

The presence of progeny bacteriophage may be determined by any of many methods well known in the art. For example, progeny bacteriophage may be detected by conventional plaque assay methods or by automated technologies, including, for example, cell sorters, such as fluorescent activated cell sorting (FACS).

Progeny bacteriophage may also be detected by direct visualization. Such direct visualization may be by light or a fluorescent microscope. Stains or enzymes that may be used include, but are not limited to, the fluorescent probe ALEXA (available from Molecular Probes, Inc., Eugene, Oreg.), Cy3, fluorescein isothiocyanate, tetramethylrhodamine, horseradish peroxidase, alkaline phosphatase, glucose oxidase or any other label known in the art.

QUANTUM DOTS nanocrystals, manufactured by Quantum Dot Corp., Hayward, Calif. may be used in the methods of the present invention to detect bacteria cells or viruses. QUANTUM DOTS are nanoscale crystals that exhibit a number of favorable characteristics over conventional fluorescent dyes. Unlike fluorescent dyes, QUANTUM DOTS nanocrystals photobleach much more slowly and fluoresce much more brightly. Because of the array of different sizes available, QUANTUM DOTS nanocrystals cover a broader optical spectrum (i.e., different sizes emit different colors), thereby allowing for the detection of different organisms in the same sample. QUANTUM DOTS nanocrystals are manufactured with the same uniform conjugational chemistry, thereby providing consistent behavior under multiple assay environments. Currently, QUANTUM DOTS nanocrystals are available as three different conjugates; streptavidin, protein A, and biotin. In some embodiments of the present invention, streptavidin conjugates may be used to fluoresce progeny bacteriophage via a QUANTUM DOT-streptavidin-biotin-antibody complex. The streptavidin conjugates are extremely bright, provide excellent photostability, and have a single excitation source.

Alternatively a laser system may be used to detect labeled bacteriophage. Other detection methods include the detection of adenylate kinase, see Murphy et al., pp. 320-322 of Bioluminescence and Chemiluminesence in Medicine and Disease, Clinical Chemistry and Microbiology, and detection using a binomial-based bacterial ice nucleation detection assay, see Irwin et al., Journal of AOAC International 83:1087-95 (2000).

Or, for some embodiments, progeny bacteriophage may also be detected by methods utilizing bioluminescence, detecting the expression of a luciferase gene cloned into the bacteriophage genome. See, for example, Loessner et al., Applied and Environmental Microbiology 62(4):1133-1140 (1996).

Bioluminescence has perhaps the highest intrinsic sensitivity among biochemical detection methods. Expression of the lux (bacterial luciferase) gene can be detected at high sensitivity by measuring the light emitted by the cells expressing the gene in the presence of a suitable substrate. Several investigators have incorporated lux into a phage genome and used the resulting phage to express lux in a target bacterium.

Although the intrinsic sensitivity of bioluminescence assays is unsurpassed, this sensitivity is often unrealized. Light detection down to the level of single photons is readily achieved, however limitations arise first in getting the emitted photons to the detector and second in distinguishing them from spurious background signals including phosphorescence. In complex samples, emitted photons are readily obscured by scattering or absorption by other sample components. Sample geometry is also a factor in efficiently delivering emitted photons to the detector. The emitted light will be most readily observed if the luciferase-expressing cells are separated from opaque components of the medium and potential sources of background and arranged in a thin layer with close optical coupling to the detector.

Elimination of Phage Background

The methods of the present invention overcome problems associated with phage background in conventional processes. For example, if one were to add one thousand phage particles to a sample containing five target bacteria, each of the five bacterial cells becomes multiply infected, and after a certain interval releases, for example, one hundred progeny phage. If these are identical to the starting phage, and if 75% of the starting phages are still present, a signal less than the background level is obtained, resulting in a very difficult detection problem. If one started with ten times as many phage or had only one cell to detect, the background would be overwhelming. But a substantial excess of phage, of the order of 10-fold greater than the number of cells, are needed for reliable and speedy detection of small numbers of bacteria. A previous approach to this problem has been to destroy the remaining extracellular phage chemically after the target cells are infected. However, the chemical treatment may kill the pathogen cells before they are able to produce new phage particles.

The methods of the present invention overcome these problems by using phage particles that include a binding agent or by immobilizing the initial phage particles. Immobilization can occur by entrapment in a film coating, or by crosslinking to a surface, polymer matrix or polymer particle. The advantage of these approaches is that it is not dependent on the efficiency of chemical inactivation, and a large excess of the initial phage can be used.

An alternative method of the present invention is to adsorb the unattached initial phage to a selective adsorbent. A suitable adsorbent can be prepared from a layer of immobilized cells of the target species. These are immobilized securely so they do not leach into the sample and cause false positives. The immobilization techniques are selected such that they do not generally interfere with the reaction of cells with the phage, but this is a less severe restriction than with immobilization of the phage since the bacteria are much larger particles with multiple recognition sites for the phage.

Preferably, the immobilized bacteria used will be inactivated or genetically modified to be non-pathogenic. Also preferably they should be protected from phage infection at some internal point, for instance a restriction system or a non-permissive mutation, to remove the potential for false positives.

Immobilized Binding Agents

An immobilized binding agent binds to the bacteriophage. An immobilized binding agent includes, but is not limited to streptavidin; an antibody that specifically binds to the bacteriophage or to a bacteriophage substructure, such as the head; an isolated viral receptor protein; and a cell that is capable of being infected by the bacteriophage. Binding agents are immobilized on a substrate by methods well known in the art.

For example, bacteriophage-specific antibodies can be immobilized on a substrate. As used herein, the term “antibodies” includes polyclonal antibodies, affinity-purified polyclonal antibodies, monoclonal antibodies, and antigen-binding fragments thereof, such as F(ab′)₂ and Fab proteolytic fragments. The term “polyclonal antibody” refers to an antibody produced from more than a single clone of plasma cells; in contrast “monoclonal antibody” refers to an antibody produced from a single clone of plasma cells. Polyclonal antibodies may be obtained by immunizing a variety of warm-blooded animals such as horses, cows, goats, sheep, dogs, chickens, rabbits, mice, hamsters, guinea pigs and rats as well as transgenic animals such as transgenic sheep, cows, goats or pigs, with an immunogen. The resulting antibodies may be isolated from other proteins by using an affinity column having an Fc binding moiety, such as protein A, or the like. Monoclonal antibodies can be obtained by various techniques familiar to those skilled in the art. Briefly, spleen cells from an animal immunized with a desired antigen are immortalized, commonly by fusion with a myeloma cell (see, Kohler and Milstein (1976) Eur. J. Immunol. 6, 511-519; J. Goding (1986) In “Monoclonal Antibodies: Principles and Practice,” Academic Press, pp 59-103).

Isolated bacteriophage, or substructures thereof, can serve as an antigen to immunize an animal to elicit an immune response. For example, antibodies to intact bacteriophage, isolated precursor bacteriophage head particles, or isolated capsid particles can be prepared.

The phrase “specifically binds” or “specifically immunoreactive with,” when referring to an antibody, refers to a binding reaction that is determinative of the presence of a protein in a heterogeneous population of proteins and other biologics. Thus, under designated immunoassay conditions, the specified antibodies bind to a particular protein at least two times the background and do not substantially bind in a significant amount to other proteins present in the sample. Typically a specific or selective reaction will be at least twice background signal or noise and more typically more than 10 to 100 times background. Specific binding to an antibody under such conditions may require an antibody that is selected for its specificity for a particular protein.

For detection methods in which the functionality of the bacteriophage must be maintained, for example the functional ability to infect a bacterial cell, antibodies with a specificity for bacteriophage tail proteins should not be used, as the binding of such an antibody to the tail proteins can interfere with the ability of the bacteriophage particle to bind to a host bacterial cell.

Immobilization of Bacteriophage on a Substrate

Bacteriophage may be immobilized on a substrate by one of many procedures known in the art. For example, an antibody specific for the bacteriophage may be used to attach a bacteriophage to a substrate. Alternatively, protein A, protein G, or ligands, such as avidin, streptavidin and biotin, may be used. Covalent linkage methods may also be used to attach a bacteriophage to a substrate.

Methods for immobilizing binding agents on a substrate are described, for example, in U.S. Pat. Nos. 5,679,510 and 6,165,710, and in Davidson et al., J. Sep. Sci. 24:10-16 (2001).

Substrates

Substrates to be used in the method for the present invention include, but are not limited to, polystyrene beads (Spherotech, Libertyville, Ill.), magnetic beads (Dynal Biotech, Lake Success, N.Y.), latex coatings, a membrane filter, a fiber filter, a free fiber or a porous solid substrate. Methods for the use of magnetic beads can be found, for example, with the package insert of Dynabeads Protein G Prod. No. 100.03/04, in Kala et al., Analytical Biochemistry 254:263-266 (1997) and in Dutton, Genetic Engineering News, Volume 22, Number 13, July 2002.

A wide spectrum of particles, particularly magnetic and polystyrene beads, are commercially available in a wide range of sizes. For certain embodiments, a preferred set of particles has an average particle size (i.e., the largest dimension of the particles) of at least 2 micrometers (i.e., microns). For certain embodiments, a preferred set of particles has an average particle size (i.e., the largest dimension of the particles) of no greater than 4 micrometers (i.e., microns).

For certain embodiments, the concentration of particles (e.g., beads) is preferably at least 1000 particles (e.g., beads) per milliliter. For certain embodiments, the concentration of particles (e.g., beads) is preferably no greater than 10,000 particles (e.g., beads) per milliliter. This size range allows for evaluation in a two-dimensional array without stacking, which facilitates observation of anything attached to the particles.

Exemplary commercially available beads are protein-G coated polystyrene beads and streptavidin-coated polystyrene beads, both available from Dynal Biotech, Lake Success, N.Y. Protein-G-coated polystyrene beads are also commercially available from Spherotech, Libertyville, Ill.

Samples

Samples include, but are not limited to, environmental or food samples and medical or veterinary samples. Samples may be liquid, solid, or semi-solid. Samples may be swabs of solid surfaces. Samples may include environmental materials, such as the water samples, or the filters from air samples or aerosol samples from cyclone collectors. Samples may be of meat, poultry, processed foods, milk, cheese, or other dairy products. Medical or veterinary samples include, but are not limited to, blood, sputum, cerebrospinal fluid, and fecal samples and different types of swabs.

Samples may be used directly in the detection methods of the present invention, without preparation or dilution. For example, liquid samples, including but not limited to, milk and juices, may be assayed directly. Samples may be diluted or suspended in solution, which may include, but is not limited to a buffered solution or a bacterial culture medium. A sample that is a solid or semi-solid may be suspending in a liquid by mincing, mixing or macerating the solid in the liquid. A sample should be maintained within a pH range that promotes bacteriophage attachment to the host bacterial cell. A sample should also contain the appropriate concentrations of divalent and monovalent cations, including but not limited to Na⁺, Mg⁺⁺, and K⁺. Preferably a sample is maintained at a temperature that maintains the viability of any pathogen cells contained within the sample.

Assay Conditions

Preferably throughout detection assays, the sample is maintained at a temperature that maintains the viability of any pathogen cell present in the sample. During steps in which bacteriophage are attaching to bacterial cells, it is preferable to maintain the sample at a temperature that facilitates bacteriophage attachment. During steps in which bacteriophage are replicating within an infected bacterial cell or lysing such an infected cell, it is preferable to maintain the sample at a temperature that promotes bacteriophage replication and lysis of the host. Such temperatures are at least about 25° C., more preferably no greater than about 45° C., most preferably about 37° C. It is also preferred that the samples be subjected to gentle mixing or shaking during bacteriophage attachment, replication and lysis.

Assays may include various appropriate control samples. For example, control samples containing no bacteriophage or control samples containing bacteriophage without bacteria may be assayed as controls for background levels.

Preferred Assays

In one embodiment, the first step is to add phage to the test sample. The target bacterial cells are infected when they come into contact with the phage. After sufficient time for infection of the bacterial cells, unreacted phage particles are removed from the sample.

Unreacted phage may be removed from the sample by the contacting the sample with a substrate to which a binding agent for the bacteriophage is immobilized. The substrate is then removed from the sample.

Infected bacterial cells are incubated under conditions to form new bacteriophage. New bacteriophage may be detected by a variety of means. For example, new bacteriophage may be detected by contacting the solution with a second substrate to which a binding agent for new bacteriophage is immobilized. New bacteriophage may be concentrated prior to contacting with a second substrate to which a binding agent for the new bacteriophage is immobilized. The presence of new bacteriophage in the sample indicates the presence of target bacterial cells in the sample and the absence of new bacteriophage indicates the absence of target bacterial cells in the sample.

In other embodiments, unreacted phage particles may be removed from solution by the reaction with immobilized reporter cells firmly attached to the surface of a dipstick. The dipstick is removed from the solution before new phage particles are released from the original pathogen cells. The new phage particles are then detected by the reporter cells immobilized in another coated strip or dipstick. Since only new phage particles are available to react with the reporter cells, there is no need to kill or inactivate the extra phage added in the initial step of the method.

In another embodiment of the invention, the first “dipstick” bearing the adsorbing cells takes the form of a disk with cells on both surfaces. This is initially placed on the surface of the sample in a petri dish or similar container, presenting a large surface and short diffusion path to the cells. As the disk sinks, the partly depleted reaction mixture flows up and over the top surface of the disk and there sees a fresh supply of adsorbent, thus being subject to a two-stage extraction with increased mass-action driving force in the later stages when the reaction would ordinarily slow down.

Once the excess phage have reacted, but before the infected target cells begin to burst, the first dipstick is removed and replaced with a second dipstick bearing target cells which are infected with the produced phage and produce luminescence or other signal detectable at high sensitivity. This can be read in situ for clear samples or removed and placed separately in the readout instrument for samples that are turbid or otherwise reduce detection efficiency.

In another embodiment, initial phage particles are immobilized in/on a patch coating covering a test strip or dipstick. The target pathogen cells are infected when they come into contact with the immobilized phage. New phage particles are produced by the pathogen cells and are released into the solution. The new phage particles are then detected by the reporter cells immobilized in another coated strip or dipstick. Both test strips may be on the same dipstick (therefore the name double dipstick). Since only new phage particles are free to react with the reporter cells there is no need to kill or deactivate the extra phage added in the initial step of the method.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Bacteriophage φ29 Precursor Capsids (Proheads) Attached to Polystyrene Beads were Active in DNA Packaging In Vitro

Bacteriophage φ29 is a small, double-stranded DNA, tailed phage of Bacillus subtilis (Anderson, et al., J. Bacteriol. 91:2081-2089, 1966; for a review, see Anderson and Reilly, In Bacillus subtilis and other Gram-Positive Bacteria: Physiology, Biochemistry and Molecular Genetics, Hoch, Losick and Sonenshein (eds.), ASM Publications, pp859-867, 1993). Precursor φ29 heads (proheads) bound to antibody-coated microspheres efficiently packaged φ29 DNA in vitro in bulk assays and in single molecule studies (for a review, see Grimes et al., Adv. Virus Res. 58:255-294, 2002).

Polystyrene microspheres coated with protein G (2.8 um diameter, 5% w/v; Spherotech, Libertyville, Ill.) were washed twice in TMS buffer (50 mM Tris-HCl (pH 7.8), 10 mM MgCl₂ 100 mM NaCl) and incubated for 20 minutes with a 1/10 dilution of rabbit antiserum prepared against bacteriophage φ29. The antibody-coated microspheres were washed five times with TMS buffer by centrifugation. Proheads were added to the microspheres to give 500 proheads per sphere, and binding occurred during 30 minutes at 4° C. with occasional mixing. Approximately 99% of the proheads bound the beads and remained attached during four washes with TMS buffer by centrifugation. The prohead-bead complexes were mixed with φ29 DNA, the DNA packaging ATPase gp16, and ATP in TMS buffer to give a ratio of 2 proheads: 1 DNA genome: 12 gp16 molecules, and each DNA molecule was packaged into a bead-bound prohead as quantified with a DNase protection assay and agarose gel electrophoresis (see Grimes and Anderson, J. Molecular Biology 209:91-100, 1989 for a more complete discussion of the assay methods).

In addition to the bulk DNA packaging assay, force-measuring laser tweezers were used to follow DNA packaging activity of a single complex in real time (see Smith et al., Nature 413:748-752, 2001). Partly prepackaged complexes, stalled in DNA packaging by the addition of the ATP analogue gammaS-ATP, were attached to polystyrene beads by means of the biotinylated, unpackaged end of the DNA. This bead was captured in the optical trap and brought into contact with a second bead held by a pipette that was coated with anti-φ29 antibodies, forming a stable tether between the beads. Shortly after addition of ATP, the beads moved closer together as a result of DNA packaging. The force-velocity relationship of the motor was established, and the motor was found to be one of the strongest molecular motors reported, working against loads of up to 57 picoNewtons.

### Example 2

### Bacteriophages were Tethered to Magnetic Polystyrene Beads Via Anti-Phage Antibodies or a Biotin-Streptavidin Linkage

a) Attachment of bacteriophage φ29 to magnetic beads via φ29-specific antibodies. Dynabeads Protein G (Cat. No. 100.03, Dynal Biotech, Lake Success, N.Y.) are magnetic polystyrene beads, 2.8 μm in diameter, coated with recombinant protein G covalently coupled to the surface. The Dynabeads are supplied in phosphate buffered saline (PBS), pH 7.4, containing 0.1% Tween-20 and 0.02% sodium azide. The density of the beads is approximately 1.3 g/cm³. The magnetic Particle Concentrator (Cat. No. MPC-S#120.20, Dynal Biotech, Lake Success, N.Y., hereafter referred to as the MPC) is used to retrieve the beads from Microcentrifuge Tubes (Cat. No. 120.20 Dynal Biotech, Lake Success, N.Y.). First, the Dynabeads were washed three times in 10 bead volumes of PBS, each time retrieving the beads with the MPC. To attach φ29 polyclonal anti-head antibodies (prepared in the rabbit against purified φ29 precursor capsids (proheads) by Rockland Immunochemicals, Inc., Gilbertsville, Pa.) to the washed beads, 15 μl PBS and 5 μl anti-φ29 antibody (IgG fraction of serum, obtained by chromatography on a protein A column, about 3 mg/ml IgG) were added to 50 μl (6.6×10⁷) beads, and the mixture was incubated at room temperature for 40 minutes with gentle rocking in a mixer (Cat. No. 947.01 Dynal Biotech, Lake Success, N.Y., hereafter referred to as the Dynal mixer). The bead-antibody complexes were retrieved with the MPC, washed once in 0.5 ml of PBS, retrieved again with the MPC, washed gently in TMS buffer (50 mM Tris-HCl, pH 7.8, 10 mM MgCl₂, 100 mM NaCl) two times, and retrieved. Finally, 2×10³ φ29 phages in TMS buffer were added per bead-antibody complex, and the mixture was incubated for one hour at 4° C. with gentle rocking in the Dynal mixer. After retrieving the bead-antibody-φ29 complexes with the MPC, the supernatant contained 30% of the input phages, demonstrating that 1.4×10³ φ29 viruses were adsorbed to each bead. After the bead-phage complexes were washed four times, each with 300 μl of TMS buffer, the supernatant contained less than 0.1% of the phages that initially adsorbed to the beads; thus the phages were quite firmly attached. The bead-antibody-φ29 complexes were resuspended in 50 μl of TMS buffer.

b) Preparation of biotin-labeled φ29 and attachment to streptavidin-coated magnetic beads. To produce biotin-labeled phage φ29, EZ-Link Sulfo-NHS-LC-Biotin (Cat. No. 21335, Pierce Biotechnologies Inc., Rockford, Ill.) was used. φ29 (3×10¹¹) in 50 μl Hepes buffer (50 mM Hepes (pH 7.5), 10 mM MgCl₂, 100 mM NaCl) was mixed with 6.14 μg (2×10⁴ biotin molecules per phage particle) of sulfo-NHS-LC-biotin and incubated overnight at 4° C. This mixture was passed through MicroSpin G50 columns (Amersham Biosciences Cat. No. 27-5330-01) twice to remove unbound biotin, and after bringing the volume to 1 ml, the φ29 titer by plaque assay was 3×10¹¹ per ml, showing complete recovery and full infectivity of the biotin-labeled particles. The presence of biotin on the surface of the particles was demonstrated by the addition of an excess of free streptavidin followed by SDS-PAGE, which detected streptavidin-biotin complexes of the major capsid protein and other φ29 structural proteins by gel shift.

To attach biotin-labeled φ29 to magnetic polystyrene beads coated with streptavidin (Cat. No. 12.05/06, Dynal Biotech), the beads (6.5×10⁴) were washed twice with Hepes buffer (50 mM Hepes (pH 7.5), 10 mM MgCl₂, 100 mM NaCl) and incubated with biotin-labeled phages (2×10³ phages per bead) with continual rotation at 17 rpm for 2 hours at room temperature. The bead-phage complexes were placed in a magnetic particle concentrator (Dynal Biotech MPC-S #120.20) to remove unbound phages and washed 3 times, each with 150 μl of Hepes buffer, and finally resuspended in 50 μl of TMS buffer. Plaque titer of the initial supernatant showed that 10³ phages were attached to each bead.

### Example 3

### Biotin-Labeled Bacteriophages Detect and Quantify Target Bacteria in a Sample by a Phage Amplification/Progeny Retrieval/Direct Count Assay

Bacteriophage φ29 of Bacillus subtilis, biotin-labeled as described in example 2, is added (10² particles) to a 1 ml sample containing B. subtilis (10 cells), together with nutrients needed for cell growth, and the sample is incubated at room temperature with gentle rocking in the Dynal mixer. At 30 minutes after infection, 3×10³ magnetic polystyrene, streptavidin-coated beads (Dynal Biotech Cat. No. 120.20) are added to bind excess biotin-labeled phages that did not adsorb to target cells (separate experiments have demonstrated that 10³ streptavidin beads can bind and remove as few as 100, 10 or even 1 biotin-labeled phage particle(s) from a 250 μl sample in 15 minutes). The infected cells are incubated at 37° C. for an additional 30 minutes with gentle rocking in the Dynal mixer to permit cell lysis. The bead-streptavidin-biotin-phage complexes are then removed from the lysate by use of the MPC, and the supernatant contains the progeny phage, which do not contain biotin and do not bind to the streptavidin-coated beads. Then 3×10² protein G magnetic beads (Dynal Biotech Cat. No. 100.03/04) coated with anti-φ29 antibodies are added and mixed with the supernatant to bind the 100 progeny particles produced per infected cell (10 cells×100 phage progeny=10³ phages on 3×10² beads=˜3 phages per bead), and the mixture is incubated with gentle rocking on the Dynal mixer for 15 minutes at ambient temperature. The mixture is added to a detachable, hollow cylinder with an inside diameter of 9 mm and a height of 1.6 mm (1 ml capacity) that is mounted on a thin (0.13 mm) coverslip over a magnetic needle. Within 10 minutes the 2.8 um beads settle to the bottom of the chamber and are concentrated on the coverslip over the tip of the magnet. The bulk of the liquid is removed with a pipete, the chamber is detached, and the last 50 μl are carefully removed via the absorption properties of a bibulous paper strip. The bead-phage complexes remain centered in a spot with a diameter of about 0.7 mm, in ˜0.25 μl of liquid, resulting in a ˜1,000-fold concentration of the beads. In separate experiments the hollow cylinder was attached to a glass coverslip, over a magnetic needle, by use of vacuum grease. 3×10³ magnetic beads were concentrated quantitatively from a 1 ml sample, and the two-dimensional array of beads was easily visualized at 160× in brightfield microscopy. The magnet is removed, and a solution of a fluorescent probe such as ALEXA 488 dye (Molecular Probes #A-10254) or QUANTUM DOTS 565 (QUANTUM DOTS Corp #003-1, applied as a φ29 antibody-biotin-streptavidin-Qdot complex) is added to label the progeny phage on the beads. Excess fluorescent tag is removed by two washes, each time using the magnetic needle to concentrate the beads, and bibulous paper is used to remove practically all of the liquid. The sample dries quickly and is observed directly by fluorescence microscopy at a magnification of 1,000×; at this magnification the 2.8 micrometer beads have an apparent size of 2.8 millimeters, and individual phage particles on beads appear as bright dots that can be counted. Individual ALEXA-labeled φ29 particles have been observed both attached to magnetic beads and free in solution by fluorescence microscopy at 1,000×, showing that ALEXA-labeled single phage particles have an apparent size roughly 10× that of the actual size of the virus. Moreover, QUANTUM DOTS, molecular scale optical nanocrystals, commercially available as streptavidin complexes and coupled to biotin-labeled anti-φ29 antibodies, are preferred over ALEXA dye because they are photo-stable and much brighter than organic dyes like ALEXA; in addition, only the phage antigen of interest will fluoresce. When ALEXA 488 is used, the protein G and antibody components on the beads stain only lightly, while the phage particles that have a mass two hundred times greater than the antibodies appear as bright stars. As indicated above, ˜3 phages per bead, on average, are observed, and the number of phage produced and captured reflects the number of target bacteria in the sample. See example 6 for a discussion of positive and negative controls needed for definitive results with this assay. The method has the potential of detecting a single target cell in a 1 ml sample, because 100 phage progeny will readily be observed and enumerated.

### Example 4

### Biotin-Lableled Bacteriophage A511 Engineered to Carry the luxAB Gene can Detect and Quantify Listeria monocytogenes in a Sample by a Phage Amplification/Immobilized Reporter Cell Assay

Bacteriophage A511 of Listeria monocytogenes has been engineered to carry the luxAB gene, which bestows the bioluminescence phenotype on infected host cells (Loessner et al., Applied and Environmental Microbiology, 62:1133, 1996). The phage is grown by standard methods and purified by isopycnic centrifugation in CsCl. The purified phage is biotin-labeled as described in Example 2. The biotin-labeled A511 phage (10²) are added to infect Listeria target cells (10) in a 1 ml sample supplemented with appropriate ions and nutrients for cell growth, and the sample is incubated at 37° C. with gentle agitation. At 30 minutes after infection, magnetic polystyrene beads coated with streptavidin (10³) are added to adsorb excess biotin-labeled input phages that have not attached to host cells. One hour after infection the infected cells lyse, each cell releasing roughly 100 phage progeny (10 cells×100 progeny per cell=10³ total phage progeny). The magnetic streptavidin beads with adsorbed biotin-labeled input phages, some attached to target cell envelopes, are removed with the MPC as described in Example 3, leaving behind the new phages replicated by the target bacteria; these progeny particles do not bind the streptavidin-coated beads. Then reporter Listeria cells (10²) carrying the luxAB gene, immobilized on magnetic polystyrene beads (10³) that are coated with protein G and anti-Listeria antibodies, are added as host cells for the new phages. A high bead/cell ratio is used to minimize bridging of beads by cells. Infection results in expression of the luxAB gene, and the bead-infected cell complexes are retrieved prior to cell lysis and concentrated by a magnetic needle over a coverslip, as described in Example 3. The bead-bound luminescent cells are counted directly in a light microscope at 160×, and this serves as a qualitative index of the presence of progeny phages, and therefore of target cells, in the sample. In addition, luminescence of the dried sample is measured in a luminometer, and a quantification of cells in the sample is obtained by reference to standards consisting of concentrated bead-immobilized bacteria that have been infected with known numbers of phage A511 carrying the lux gene. Assay of luminescent reporter cells infected with phage progeny produced by target bacteria can potentially be extrapolated to the presence of one or a few cells in a 1 ml sample.

### Example 5

### Biotin-Labeled Bacteriophages Tethered to Streptavidin-Coated Magnetic Beads can Infect and Quantify Target Bacteria in a Sample by a Phage Amplification/Bead Retrieval/Direct Count Assay

The delayed lysis mutant sus14(1241) of bacteriophage φ29 of Bacillus subtilis, which has an extended life cycle of about 120 minutes at 37° C., compared to a 35 minute life cycle for wild-type φ29 (Anderson and Reilly, J. Virol. 13:211-221, 1974), was biotin-labeled and complexed with magnetic polystyrene, streptavidin-coated beads as described in Example 2. The bead-phage complex (4×10⁶) was mixed with Bacillus subtilis (10³) in phage growth medium to give a volume of 100 μl, and the mixture was incubated under rotation at 17 rpm for 1 hour at room temperature. Next the volume was brought up to 1 ml by the addition of phage growth medium, and incubation was continued with shaking at 200 rpm for 2 hours at 37° C. Then lysozyme was added to a final concentration of 20 μg/ml to assure lysis of infected cells, and incubation was continued with shaking for an additional 20 minutes. The phage titer by plaque count demonstrated a yield of 284±162 phage per cell (2.84±1.62×10⁵ phage per ml).

This represents the first demonstration that virus particles immobilized on a substrate such as a magnetic bead can productively infect target cells. This novel method of using viruses immobilized on the surface of a retrievable substrate to infect target cells circumvents the necessity of removing excess input viruses, the major limitation of all prior phage amplification assays. Then the progeny phages can be retrieved efficiently by the use of magnetic polystyrene beads coated with anti-viral antibodies, complexed with ALEXA dye or Quantum Dots, concentrated by the use of a magnetic needle, and quantified by direct counts in the fluorescence microscope as described in Example 3.

### Example 6

### Commercial Kits for Detection and Quantification of a) Bacteria by Phage Amplification and b) Bacteria or Viruses by Direct Retrieval and Counts

Using the materials and methods described in Examples 1-5, commercial kits for the detection and quantification of bacteria by bacteriophage amplification and the direct detection and quantification of bacteria or viruses will be prepared. Printed instructions for use may also be provided in each kit.

a) A Kit for Bacteriophage Amplification for Detection and Quantification of Bacteria may Include One or More of the Following:

1) aliquots of 10³ biotin-labeled bacteriophage particles specific for the microbe of interest, in 25 μl, frozen;

2) bacteriological growth medium with and without 10² cells of the microbe of interest, which may serve as positive and negative controls in the assay, 1 ml each, frozen;

3) 10× bacteriological growth medium, 100 μl aliquots, frozen;

4) aliquots of 3×10³ 2.8 μm diameter magnetic polystyrene, streptavidin-coated beads, in 25 μl, refrigerated;

5) aliquots of 3×10³ 2.8 μm diameter magnetic, polystyrene, antibody-coated beads, 25 μl, refrigerated;

6) aliquots of biotin-antibody-streptavidin-Quantum Dot complex, 25 μl, refrigerated;

7) thin coverslip slide with detachable hollow cylinders mounted over magnetic needles; and

8) bibulous paper strips.

The printed instructions that may be provided with a kit may include some or all of the following instructions. The biotin-labeled bacteriophage are added to the cultures of the positive and negative controls as well as the unknown sample, the latter is fortified with 1/10^{th} volume of 10× growth medium, and the mixtures are incubated at 37° C. for 15 minutes with gentle rocking on the Dynal mixer. Magnetic streptavidin-coated beads are added to the cultures to adsorb excess biotin-labeled phages that have not attached to host cells (infected host cells may also attach to these beads via surface phages, but this is of no consequence). After 1 hour the infected cells of the cultures lyse, each liberating about 100 phage progeny, and the streptavidin beads with adsorbed phages (some complexed to lysed cell envelopes) are removed with the MPC. To the supernatants are added the magnetic antibody-coated beads, the mixtures are incubated for 15 minutes while the beads adsorb the progeny phages, and the beads are concentrated to 0.7 mm spots by use of the detachable hollow cylinders mounted on the coverglass over the magnetic needles (as described in Example 3). Then the bead-adsorbed phages are labeled with Qdot complexes and the beads concentrated for fluorescence microscopy and direct counts of phages as described in Example 3 (it has been demonstrated that 3×10³ beads in 1 ml are recovered essentially quantitatively with the magnet, that they form a two-dimensional array without stacking, and that all of the beads are visible in one microscope field at a magnification of 160×). The procedure takes 1.5-2 hours and can potentially detect a single cell in a volume of 1 ml since the 100 progeny of one target cell are readily observed and enumerated.

b) Kits for Direct Counts of Bacteria or Viruses may Include One or More of the Following:

1) aliquots of 3×10³ 2.8 μm diameter magnetic polystyrene beads coated with protein G and complexed with specific antibody against the agent of interest, in 25 μl, refrigerated;

2) buffer with or without 10² cells or viruses of interest, which may serve as positive and negative controls, 1 ml each, frozen;

3) thin coverslip slide with detachable hollow cylinders mounted over magnetic needles;

4) aliquots of biotin-antibody-streptavidin-Quantum Dot complex, 25 μl, frozen; and

5) bibulous paper strips.

The written instructions that may be provided with a kit may include some or all of the following instructions. The magnetic beads coated with specific antibody are added to the samples of the positive and negative controls as well as to the unknown sample (1 ml), and the mixtures are incubated with gentle rocking on the Dynal mixer at 37° C. for 30 minutes. The mixtures are transferred to the hollow cylinders mounted to the coverglass over the magnetic needles, the beads with the attached agent are concentrated within areas with diameters of about 0.7 mm, and the supernatants are drawn away with a pipete and bibulous paper strips. Then the bead-adsorbed agents are labeled with Qdot complex, and the beads are concentrated for fluorescence microscopy and direct counts of the agent as described in Example 3. The procedure takes 1.5-2 hours and has the potential of detecting a single bacterial cell or virus in a volume of 1 ml.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. The foregoing detailed description and examples have been given for clarity of understanding only. No unnecessary limitations are to be understood there from. The invention is not limited to the exact details shown and described, for variations obvious to one skilled in the art will be included within the invention defined by the claims.

What is claimed is:

1. A method of detecting a bacterial cell in a sample comprising:
contacting the sample with bacteriophage comprising a binding agent, wherein the bacteriophage is specific to the bacterial cell;
incubating the sample under conditions effective for the bacteriophage comprising a binding agent to infect the bacterial cell, and if a bacterial cell is present in the sample and has been infected by the bacteriophage comprising a binding agent, form new bacteriophage and release new bacteriophage into the sample, wherein the new bacteriophage do not comprise a binding agent;
contacting the sample with a substrate comprising immobilized ligand for the binding agent under conditions effective for a complex to form between the bacteriophage comprising a binding agent and the substrate comprising immobilized ligand for the binding agent;
removing the complexes of bacteriophage comprising a binding agent and substrate comprising immobilized ligand from the sample; and
detecting new bacteriophage in the sample from which complexes have been removed, wherein the presence of new bacteriophage indicates the presence of a bacterial cell specific for the bacteriophage in the sample and wherein the absence of new bacteriophage indicates the absence of a bacterial cell specific for the bacteriophage in the sample.

2. The method of claim 1 wherein the bacterial cell is a food pathogen.

3. The method of claim 2 wherein the food pathogen is selected from the group consisting of Listeria monocytogenes, Salmonella spp., Campylobacter spp., and E. coli O157/H7.

4. The method of claim 1 wherein the bacterial cell is a medical or veterinary pathogen or a bacteria cell of commercial significance.

5. The method of claim 1 wherein the binding agent is biotin and the ligand for the binding agent is streptavidin.

6. The method of claim 1 wherein the substrate is selected from the group consisting of a polystyrene bead, a magnetic bead, a polymeric material, and combinations thereof.

7. The method of claim 1 wherein the new bacteriophage is detected using a fluorescent dye or fluorescent nanocrystals.

8. The method of claim 7 wherein new bacteriophage is detected by visualization under a light microscope.

9. The method of claim 1 wherein the substrate comprises a filter, fiber, a porous membrane, or combinations thereof.

10. A method of detecting a bacteriophage or virus in a sample comprising:
contacting the sample with a substrate comprising immobilized binding agent specific for the bacteriophage or virus, under conditions effective for a complex to form between the bacteriophage or virus and the substrate comprising immobilized binding agent for the bacteriophage or virus;
removing the complexes of bacteriophage or virus and substrate comprising immobilized binding agent for the bacteriophage or virus; and
detecting the complexes of bacteriophage or virus and substrate comprising immobilized binding agent for the bacteriophage or virus, wherein the presence of complexes of bacteriophage or virus and substrate comprising immobilized binding agent for the bacteriophage or virus indicates the presence of a bacteriophage or virus in the sample and wherein the absence of complexes of bacteriophage or virus and substrate comprising immobilized binding agent for the bacteriophage or virus indicates the absence of bacteriophage or virus in the sample.

11. The method of claim 10, wherein the binding agent for the bacteriophage or virus is an antibody that binds to the bacteriophage or virus.

12. The method of claim 10, wherein the substrate comprising immobilized binding agent specific for the new bacteriophage or virus comprises fluorescent nanocrystals.

13. A method of detecting a bacterial cell in a sample comprising:
contacting the sample with bacteriophage comprising a binding agent, wherein the bacteriophage is specific to the bacterial cell;
incubating the sample under conditions effective for the bacteriophage comprising a binding agent to infect the bacterial cell, and if a bacterial cell is present in the sample and has been infected by the bacteriophage comprising a binding agent, form new bacteriophage and release new bacteriophage into the sample, wherein the new bacteriophage do not comprise a binding agent;
contacting the sample with a substrate comprising immobilized ligand for the binding agent under conditions effective for a complex to form between the bacteriophage comprising a binding agent and the substrate comprising immobilized ligand for the binding agent;
removing the complexes of bacteriophage comprising a binding agent and substrate comprising immobilized ligand from the sample;
contacting the sample with a second substrate comprising immobilized binding agent specific for the new bacteriophage, under conditions effective for a complex to form between the new bacteriophage and the second substrate comprising immobilized binding agent for the new bacteriophage;
removing the complexes of new bacteriophage and second substrate comprising immobilized binding agent for the new bacteriophage; and
detecting the complexes of new bacteriophage and second substrate comprising immobilized binding agent for the new bacteriophage, wherein the presence of complexes of new bacteriophage and second substrate comprising immobilized binding agent for the new bacteriophage indicates the presence of a bacterial cell specific for the bacteriophage in the sample and wherein the absence of complexes of new bacteriophage and second substrate comprising immobilized binding agent for the new bacteriophage indicates the absence of a bacterial cell specific for the bacteriophage in the sample.

14. The method of claim 13, wherein the binding agent for the new bacteriophage is an antibody that binds to the new bacteriophage.

15. The method of claim 13, wherein the binding agent is biotin and the ligand for the binding agent is streptavidin.
